(19) United States
(12) Patent Application Publication
(10) Pub. No.: US 2013/0059369 A1
(43) Pub. Date: Mar. 7, 2013

(54) MICROALGAE CULTIVATION MODULE

(71) Applicants: Chih-Sheng Lin, Hsinchu City (TW); Sheng-Yi Chiu, Hsinchu City (TW); Chien-Ya Kao, Hsinchu City (TW)

(72) Inventors: Chih-Sheng Lin, Hsinchu City (TW); Sheng-Yi Chiu, Hsinchu City (TW); Chien-Ya Kao, Hsinchu City (TW)

(73) Assignee: NATIONAL CHIAO TUNG UNIVERSITY, Hsinchu City (TW)

(21) Appl. No.: 13/351,238

(22) Filed: Jan. 17, 2012

(30) Foreign Application Priority Data
Sep. 6, 2011 (TW) 100137168

(51) Int. Cl. C12M 1/42

(52) U.S. Cl. 435/292.1

(57) ABSTRACT

A microalgae cultivation module for carbon reduction and biomass production is provided, which includes a first photobioreactor set, a second photobioreactor set, a gas switching device and a control unit. The gas switching device is communicated to the first and the second photobioreactor sets. The control unit is coupled to and controls the gas switching device, thereby aerating a waste gas into the first photobioreactor set and aerating air into the second photobioreactor set for a first predetermined time, then aerating the waste gas into the second photobioreactor set and aerating the air into the first photobioreactor set for a second predetermined time. The first and the second photobioreactor sets include a microalgae species.

### CROSS-REFERENCE TO RELATED APPLICATION

This application claims the priority benefit of Taiwan application serial no. 100137168, filed on Sep. 6, 2011. The entirety of the above-mentioned patent application is hereby incorporated by reference herein and made a part of this specification.

### BACKGROUND OF THE INVENTION

1. Field of the Invention

The invention relates to a microalgae cultivation module and more particularly to a microalgae cultivation module for carbon reduction and biomass production.

2. Description of Related Art

In recent years, the world has been facing severe impact on environment and ecology deteriorated by greenhouse effect. The carbon emission of each person per year in Taiwan is more than four times of the average carbon emission of each person in the world. Thus, it is important to reduce the carbon dioxide (CO₂) emission generated from households and industries effectively.

A common CO₂ fixation method includes a chemical absorption method, a physical storage method, and a biological carbon fixation method. The biological carbon fixation method comprises methods such as afforestation, microalgae cultivation, and so on. Here, the technique using microalgae cultivation to reduce CO₂ is one of the most efficient biological carbon fixation methods, where the carbon fixation effect thereof can be dozens of times of that in afforestation. Microalgae have a compact volume, high photosynthesis efficiency, high growth rate, and can be easily cultivated. The microalgae obtained from the cultivation are not only adopted as food or nutrient additives, but are also used as a raw material for bioenergy. Microalgae thus have high practical application value.

However, although the efficiency of carbon fixation by microalgae is high, when the concentration of CO₂ in the waste gas exceeds the tolerance level of the microalgae species, microalgae then often result in growth retardation or death. In addition, in the microalgae cultivation module in an outdoor field, poor design of the photobioreactors and the cultivation module usually leads to poor growth of the microalgae species and inefficient contact of the microalgae species to CO₂, so that a large scale carbon reduction with high efficiency can not be achieved. Accordingly, the development of a microalgae cultivation module with higher efficiency and capable of achieving carbon reduction with high efficiency and high density cultivation has to be carried out.

### SUMMARY OF THE INVENTION

The invention is directed to a microalgae cultivation module for carbon reduction with high efficiency and high density cultivation.

The invention is directed to a microalgae cultivation module including a first photobioreactor set, a second photobioreactor set, a gas switching device, and a control unit. The gas switching device communicates to the first photobioreactor set and the second photobioreactor set. The control unit is coupled to and controls the gas switching device so as to aerate a waste gas into the first photobioreactor set and aerate air into the second photobioreactor set for a first predetermined time, and aerate the waste gas into the second photobioreactor set and aerate the air into the first photobioreactor set for a second predetermined time. Herein, the first photobioreactor set and the second photobioreactor set include a microalgae species.

According to an embodiment of the invention, the gas switching device includes a gas switcher communicating to the first photobioreactor set and the second photobioreactor set.

According to an embodiment of the invention, the gas switching device includes a first gas switcher and a second gas switcher. The first gas switcher communicates to the first photobioreactor set and the second gas switcher communicates to the second photobioreactor set.

According to an embodiment of the invention, the first photobioreactor set and the second photobioreactor set respectively include, for example, at least one photobioreactor.

According to an embodiment of the invention, the first photobioreactor set and the second photobioreactor set respectively include a photobioreactor array constituted by a plurality of photobioreactors connected in parallel, for example.

According to an embodiment of the invention, the microalgae cultivation module further includes an air supply device directly or indirectly communicates to the first photobioreactor set and the second photobioreactor set.

According to an embodiment of the invention, the microalgae cultivation module further includes a waste gas supply device communicating to the gas switching device.

According to an embodiment of the invention, the microalgae cultivation module further includes a desulfurization device configured to remove a sulfur containing gas in the waste gas before the waste gas is supplied to the first photobioreactor set and the second photobioreactor set.

According to an embodiment of the invention, the microalgae cultivation module further includes a sensor system configured to detect a content of at least one gaseous component in the first photobioreactor set and the second photobioreactor set.

According to an embodiment of the invention, the control unit controls a switching method of the gas switching device in a continuous time series generated by alternating the first predetermined time and the second predetermined time.

In light of the foregoing, by disposing two photobioreactor sets in the microalgae cultivation module in cooperation with the gas switching device operated using the control unit in the invention, the waste gas and air can be aerated into each photobioreactor set alternately. This intermittent gas supply method prevents poor growth of the microalgae species resulted from contacting the waste gas containing highly concentrated carbon dioxide for too long. Also, the waste gas can be aerated into the microalgae cultivation module continuously for carbon reduction while achieving carbon reduction with high efficiency and microalgae cultivation with high density.

In order to make the aforementioned and other features and advantages of the invention more comprehensible, several embodiments accompanied with figures are described in detail below.

### BRIEF DESCRIPTION OF THE DRAWINGS

The accompanying drawings are included to provide further understanding, and are incorporated in and constitute a part of this specification. The drawings illustrate embodiments and, together with the description, serve to explain the principles of the invention.

FIG. 1 is a schematic diagram illustrating a microalgae cultivation module according to one embodiment of the invention.

FIG. 2 is a schematic diagram illustrating a microalgae cultivation module according to another embodiment of the invention.

### DESCRIPTION OF EMBODIMENTS

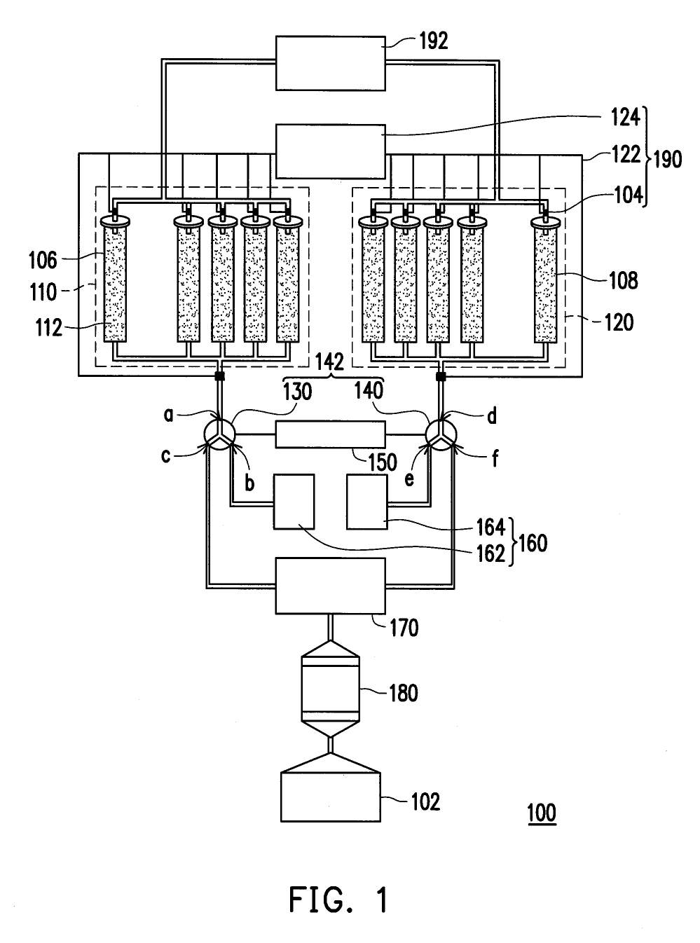

FIG. 1 is a schematic diagram illustrating a microalgae cultivation module according to one embodiment of the invention.

Referring to FIG. 1, a microalgae cultivation module 100 in the invention includes a first photobioreactor set 110, a second photobioreactor set 120, a gas switching device 142, and a control unit 150. Here, the first photobioreactor set 110 and the second photobioreactor set 120 include a microalgae species 112 for carbon fixation. The microalgae cultivation module 100 can undergo large scale carbon reduction and microalgae cultivation in an outdoor field.

The strain of the microalgae species 112 includes, for example, Chlorella, Nannochloropsis, Spirulina, Isochrysis, Synechococcus, or Haematococcus. The microalgae species 112 is favorably a strain having higher tolerance to methane (CH₄) and carbon dioxide (CO₂).

The first photobioreactor set 110 can be an array constituted by a plurality of photobioreactors 106 connected in parallel. The second photobioreactor set 120 can be an array constituted by a plurality of photobioreactors 108 connected in parallel. However, the invention is not limited thereto. In other embodiments, the first photobioreactor set 110 and the second photobioreactor set 120 can also have other arrangements, for instance, the photobioreactors can be connected in series or connected in series and in parallel. Additionally, the number of the photobioreactors 106 and the photobioreactors 108 are respectively 2 to 100, for example. The number of the photobioreactors is not specified particularly and can be adjusted depending on needs.

The photobioreactors 106 and the photobioreactors 108 are, for instance, airlift photobioreactors. An illumination source of the photobioreactors 106 is, for example, solar light or an artificial light source such as fluorescent lamps, light emitting diodes, and so on. The airlift photobioreactors are, for example, enclosed bioreactors having a shape of a hollow cylinder and with superior light permeability. The airlift photobioreactors can be used to store an incubation solution for incubating the microalgae species. A bottom of each of the bioreactors has one or more inlets and is disposed with a device for miniaturizing gas. A top of each of the bioreactors can be enclosed and disposed with a gas collection hole. For example, gases collected in the photobioreactor set can be transported to a gas collection device 192 through the gas collection hole to undergo a subsequent gas exhaustion or utilization.

The gas switching device 142 includes a first gas switcher 130 and a second gas switcher 140; however, the invention is not limited thereto. The first gas switcher 130 communicates to the first photobioreactor set 110 and the second gas switcher 140 communicates to the second photobioreactor set 120. The first gas switcher 130 has an outlet end a, an inlet end b, and an inlet end c, for instance. The outlet end a communicates to the first photobioreactor set 110. The inlet end b and the inlet end c communicate to an air supply device 160 and a waste gas supply device 170 respectively. The second gas switcher 140 has an outlet end d, an inlet end e, and an inlet end f, for instance. The outlet end d communicates to the second photobioreactor set 120. The inlet end e and the inlet end f communicate to an air supply device 160 and a waste gas supply device 170 respectively. The first gas switcher 130 and the second gas switcher 140 are electromagnetic automatic gas switching devices, for instance. Nevertheless, the invention is not limited thereto, the first gas switcher 130 and the second gas switcher 140 can also be gas switching devices of other structures known to those with common knowledge in the art.

The control unit 150 is coupled to and controls the gas switching device 142 so as to aerate a waste gas into the first photobioreactor set 110 and aerate air into the second photobioreactor set 120 for a first predetermined time, and aerate the waste gas into the second photobioreactor set 120 and aerate air into the first photobioreactor set 110 for a second predetermined time. The control unit 150 is, for example, a computer or a timer control unit; however, the invention is not limited thereto.

The control unit 150 controls a switching method of the gas switching device 142 in a continuous time series generated by alternating the first predetermined time and the second predetermined time. The first predetermined time and the second predetermined time are respectively 30 minutes, for example. Nonetheless, the length of the first predetermined time and the second predetermined time can be the same or different, and can be adjusted depending on needs.

The microalgae cultivation module 100 further includes the air supply device 160. The air supply device 160 includes an air supplier 162 and an air supplier 164. The air supplier 162 communicates to the inlet end b of the first gas switcher 130. The air supplier 164 communicates to the inlet end e of the second gas switcher 140. Accordingly, the first gas switcher 130 and the second gas switcher 140 indirectly communicates the air supply device 160 to the first photobioreactor set 110 and the second photobioreactor set 120 so as to supply air to the first photobioreactor set 110 and the second photobioreactor set 120. The air supplier 162 and the air supplier 164 are air compressors, for example. However, the invention is not limited thereto. In the present embodiment, the air supplier 162 and the air supplier 164 supply air to the first photobioreactor set 110 and the second photobioreactor set 120 respectively for illustration. However, the invention is not limited thereto. In other embodiments, the air supply device 160 can only include one air supplier, where this single air supplier supplies air to the first photobioreactor set 110 and the second photobioreactor set 120.

The microalgae cultivation module 100 further includes the waste gas supply device 170 which communicates to the inlet end c of the first gas switcher 130 and the inlet end f of the second gas switcher 140. Accordingly, the waste gas is supplied to the first photobioreactor set 110 and the second photobioreactor set 120. The waste gas supply device 170, for example, is a single water gas blower capable of supplying the waste gas to the first photobioreactor set 110 and the second photobioreactor set 120 respectively; however, the invention is not limited thereto. The waste gas supply device 170 can also include a plurality of waste gas blowers for supplying the waste gas to the first photobioreactor set 110 and the second photobioreactor set 120 respectively. The waste gas can be biogas, biogas after desulfurization, biogas after desulfurization mixed with air, or waste gases generated from various households and industries.

Moreover, the microalgae cultivation module 100 further includes a desulfurization device 180 configured to remove a sulfur containing gas in the waste gas before the waste gas is supplied to the first photobioreactor set 110 and the second photobioreactor set 120. The sulfur containing gas is hydrogen sulfide (H₂S), for example. Since H₂S inhibits the growth of the microalgae species, when processing a waste gas containing H₂S, such as biogas from a livestock farm, the concentration of H₂S in the waste gas must be decreased using the desulfurization device 180. Here, the concentration is decreased to less than 100 ppm favorably.

The desulfurization device 180 is disposed between the waste gas supply device 170 and a waste gas source 102, so that a waste gas generated from the waste gas source 102 passes through the desulfurization device 180 first to remove the sulfur containing gas. The processed waste gas is then transported to the waste gas supply device 170 and is supplied to the first photobioreactor set 110 and the second photobioreactor set 120 through the first gas switcher 130 and the second gas switcher 140 respectively. In practice, the disposition of the waste gas supply device 170 and the desulfurization device 180 is not limited thereto.

Moreover, the microalgae cultivation module 100 further includes a sensor system 190. As shown in FIG. 1, the sensor system 190 includes a host 124, a wiring 122, and a plurality of sensors 104. The sensors 104 are disposed close to the gas collection hole of each of the photobioreactors 106 and the photobioreactors 108 to detect a content of at least one gaseous component in the first photobioreactor set 110 and the second photobioreactor set 120. The wiring 122 couples to each of the sensors 104 to transmit the information sensed by the sensors 104 to the host 124 for subsequent analysis. The host 124 is, for instance, a gas detector. Nonetheless, the invention is not limited thereto. In practice, the sensor system 190 can also be in other modes. The at least one gaseous component is methane (CH₄), carbon dioxide (CO₂), hydrogen sulfide (H₂S), and a combination thereof, for example. The sensor system 190 monitors and records various gaseous components in the photobioreactors to evaluate the performance of each of the photobioreactors or each of the photobioreactor sets.

Accordingly, by disposing two photobioreactor sets in the microalgae cultivation module in cooperation with the gas switching device operated using the control unit in the invention, the waste gas and air can be aerated into each photobioreactor set alternately. This intermittent gas supply method prevents poor growth of the microalgae species resulted from contacting the waste gas containing highly concentrated carbon dioxide for too long. Also, the waste gas can be aerated into the microalgae cultivation module continuously for carbon reduction while achieving carbon reduction with high efficiency and microalgae cultivation with high density.

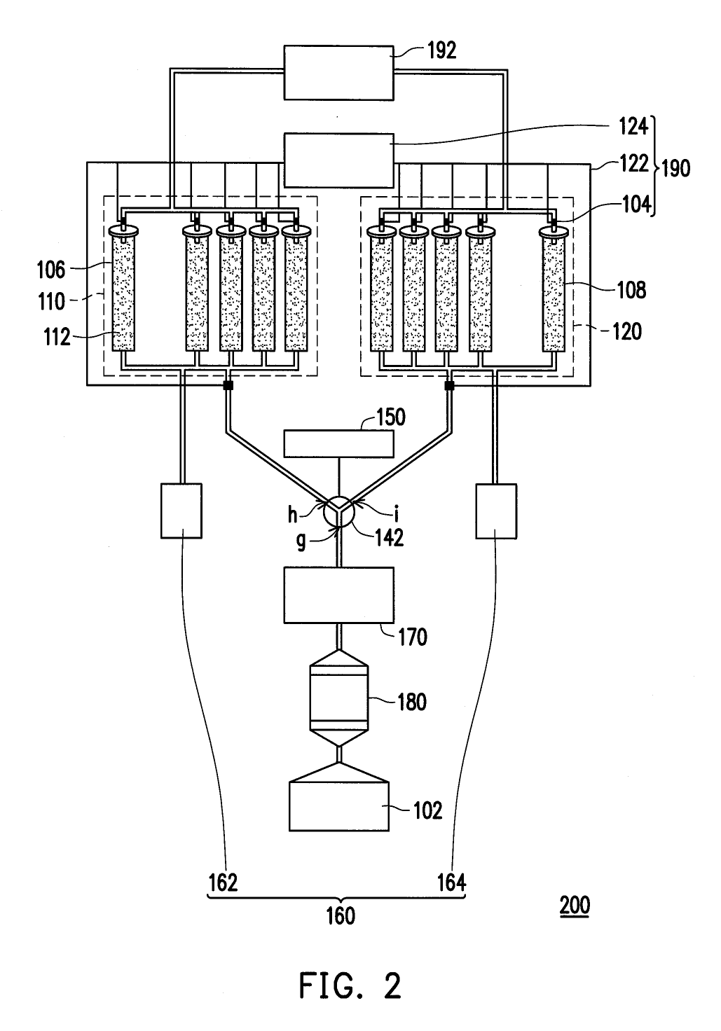

FIG. 2 is a schematic diagram illustrating a microalgae cultivation module according to another embodiment of the invention.

Referring to FIG. 2, a microalgae cultivation module 200 in the invention includes a first photobioreactor set 110, a second photobioreactor set 120, a gas switching device 142, and a control unit 150. Here, the first photobioreactor set 110 and the second photobioreactor set 120 include a microalgae species 112 for carbon fixation. An air supply device 160 includes an air supplier 162 and an air supplier 164.

The present embodiment is different from the aforementioned embodiment in that the gas switching device 142 is merely a gas switcher having an inlet end g, an outlet end h, and an outlet end i, for example. The outlet end h communicates to the first photobioreactor set 110. The outlet end i communicates to the second photobioreactor set 120. The inlet end g communicated to a waste gas supply device 170. However, the invention is not limited thereto.

In cooperation with a communication method of the gas switching device 142, the waste gas supply device 170, the first photobioreactor set 110, and the second photobioreactor set 120 in the present embodiment, the air supplier 162 directly communicates to the first photobioreactor set 110 and the air supplier 164 directly communicates to the second photobioreactor set 120 in the present embodiment. Therefore, air can be supplied to the first photobioreactor set 110 and the second photobioreactor set 120 without utilizing the gas switching device 142.

Moreover, the technical contents, device structures, characteristics and functions of the microalgae cultivation module provided in the present embodiment are described in details in the embodiment aforementioned and thus not reiterated hereinafter.

An operation method and functions of the microalgae cultivation module in the invention are illustrated in details in the following.

Accordingly, in the device of the invention, a photobioreactor set is constituted by a plurality of photobioreactors connected in parallel. A plurality of photobioreactor sets then constitutes a microalgae cultivation module. A microalgae cultivation is then carried out in an outdoor field. For example, the microalgae are used for decreasing the concentration of CO₂ in desulfurized biogas in a wastewater fermentation tank of a livestock farm. In one experimental example, a photobioreactor set is communicated to a timer-automatic gas switching device. Here, an inlet end of the gas switching device communicates to a desulfurized biogas collection bag or an air compressor. An outlet end of the gas switching device communicates to an inlet of the photobioreactor. Then, the gas switching device is operated for 8 hours (from 9:00 to 17:00) continuously in daytime with sunshine using an intermittent supply method, for example, to exchange the gas supply every 30 minutes so as to direct the desulfurized biogas from the livestock farm and air into the photobioreactors containing the microalgae alternately. That is, when the supply of the desulfurized biogas has stopped, the gas switching device then switches and directs air into the photobioreactors so as to maintain the fluctuation of the microalgae cultivation solution in the photobioreactors. In addition, since biogas and air are supplied alternately in a continuous manner, CO₂ in biogas is first dissolved and absorbed by the incubation solution in the photobioreactors, where air is then switched to for the microalgae in the photobioreactors to have sufficient time and metabolize the CO₂ dissolved in the incubation solution. This method is referred as an intermittent biogas aeration cultivation method. In this experimental example, the CO₂ content in the desulfurized biogas is measured to be about 25% at the inlet end before the processing. However, after 8 hours of processing in the photobioreactors containing the microalgae, the CO₂ content measured at the outlet end is reduced to about 12%. In other words, using the intermittent aeration method, the efficiency of removing CO₂ in biogas by the microalgae consistently can be maintained to be higher than 50%. However, when the desulfurized biogas is directed into the photobioreactor set in a single continuous aeration method (that is, aerating the desulfurized biogas into the photobioreactors containing the microalgae directly for 8 hours of processing), the efficiency of removing CO₂ in biogas is only about 10%. In terms of the growth of the microalgae species, in the present experimental example, when an 8 hour processing is performed during daylight, a growth rate of the microalgae reaches more than 0.25 g/L/d when the microalgae cultivation is performed for 5 continuous days (from 9:00 to 17:00) with intermittent aeration. This value is double of a growth rate of the microalgae cultivated using the single continuous aeration method.

In summary, by disposing two photobioreactor sets in the microalgae cultivation module in cooperation with the gas switching device operated using the control unit in the invention, the waste gas for carbon reduction and air can be aerated into each photobioreactor set alternately. The photobioreactors having the waste gas aerated in then absorbs and dissolves CO₂. The photobioreactors aerated with air then perform carbon fixation for the microalgae species to metabolize CO₂. With these alternating processes, poor growth of the microalgae species resulted from contacting the waste gas containing highly concentrated CO₂ for too long is prevented. Also, the waste gas can be aerated into the microalgae cultivation module continuously for carbon reduction while achieving carbon reduction with high efficiency and microalgae cultivation with high density.

Further, the microalgae cultivation module in the invention not only lowers the CO₂ content in the waste gas, but also increases the purity of CH₄ in the waste gas, thereby facilitating the efficiency of applying the waste gas after carbon reduction in combustion or power generation subsequently.

Also, the fat in the cultivated microalgae species undergoes transesterification to become biodiesel. The carbohydrate (including cellulose) in the microalgae species is a raw material for manufacturing bioethanol and can thus be applied in the biomass industry. Moreover, since the cells in the microalgae species are rich in protein, fatty acids, and vitamins, the microalgae species can be applied in live feed, livestock feed additive, food additive, and so on, and have high economical value.

It will be apparent to those skilled in the art that various modifications and variations can be made to the structure of the disclosed embodiments without departing from the scope or spirit of the invention. In view of the foregoing, it is intended that the invention cover modifications and variations of this invention provided they fall within the scope of the following claims and their equivalents.

What is claimed is:

1. A microalgae cultivation module, comprising:
a first photobioreactor set;
a second photobioreactor set;
a gas switching device communicating to the first photobioreactor set and the second photobioreactor set; and
a control unit coupled to and controlling the gas switching device so as to aerate a waste gas into the first photobioreactor set and aerate air into the second photobioreactor set for a first predetermined time, and aerate the waste gas into the second photobioreactor set and aerate the air into the first photobioreactor set for a second predetermined time,
wherein the first photobioreactor set and the second photobioreactor set comprise a microalgae species.

2. The microalgae cultivation module as claimed in claim 1, wherein the gas switching device comprises a gas switcher communicating to the first photobioreactor set and the second photobioreactor set.

3. The microalgae cultivation module as claimed in claim 1, wherein the gas switching device comprises:
a first gas switcher communicating to the first photobioreactor set; and
a second gas switcher communicating to the second photobioreactor set.

4. The microalgae cultivation module as claimed in claim 1, wherein the first photobioreactor set and the second photobioreactor set respectively comprise at least one photobioreactor.

5. The microalgae cultivation module as claimed in claim 1, wherein the first photobioreactor set and the second photobioreactor set respectively comprise a photobioreactor array constituted by a plurality of photobioreactors connected in parallel.

6. The microalgae cultivation module as claimed in claim 1, further comprising an air supply device directly or indirectly communicating to the first photobioreactor set and the second photobioreactor set.

7. The microalgae cultivation module as claimed in claim 1, further comprising a waste gas supply device communicating to the gas switching device.

8. The microalgae cultivation module as claimed in claim 1, further comprising a desulfurization device configured to remove a sulfur containing gas in the waste gas before the waste gas is supplied to the first photobioreactor set and the second photobioreactor set.

9. The microalgae cultivation module as claimed in claim 1, further comprising a sensor system configured to detect a content of at least one gaseous component in the first photobioreactor set and the second photobioreactor set.

10. The microalgae cultivation module as claimed in claim 1, wherein the control unit controls a switching method of the gas switching device in a continuous time series generated by alternating the first predetermined time and the second predetermined time.
